(19) United States
(12) Patent Application Publication
(10) Pub. No.: US 2025/0064429 A1
(43) Pub. Date: Feb. 27, 2025

(54) CATHETER SHAFT AND METHOD OF ITS MANUFACTURE

(71) Applicant: St. Jude Medical, Cardiology Division, Inc., St. Paul, MN (US)

(72) Inventors: Doug Manogue, Minneapolis, MN (US); John Matejka, Plymouth, MN (US)

(21) Appl. No.: 18/806,186

(22) Filed: Aug. 15, 2024

Related U.S. Application Data
(60) Provisional application No. 63/520,806, filed on Aug. 21, 2023.

(51) Int. Cl. A61B 8/00; A61B 8/08; A61B 8/12; A61M 25/00; A61M 25/01

(52) U.S. Cl. CPC A61B 8/445; A61B 8/0883; A61B 8/12; A61M 25/0009; A61M 25/0012; A61M 25/0026; A61M 25/0045; A61M 25/005; A61M 25/0136; A61M 25/0147

(57) ABSTRACT

A shaft for an elongate medical device, such as an intracardiac echocardiography or other cardiac catheter, includes a unitary inner liner defining a central lumen and one or more satellite lumens positioned about a periphery of the central lumen and an outer layer bonded to the inner liner. The wall of the inner liner is between about 0.004 inches and about 0.006 inches thick where the inner liner separates the central lumen from each of the one or more satellite lumens and may have a similar thickness at other locations as well. Each satellite lumen can accommodate a pull wire, and the number and positioning of the satellite lumens about the periphery of the central lumen can facilitate deflection of the distal end of the shaft in various directions and planes.

### CROSS-REFERENCE TO RELATED APPLICATIONS

This application claims the benefit of U.S. provisional application No. 63/520,806, filed 21 Aug. 2023, which is hereby incorporated by reference as though fully set forth herein.

### BACKGROUND

The present disclosure relates generally to elongate medical devices that are used in the human body. More particularly, the present disclosure relates to the construction of shafts for steerable medical devices, such as steerable electrophysiology (EP) and intracardiac echocardioagraphy (ICE) catheters.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

Those of ordinary skill in the art will be familiar with steerable medical devices. In such catheters, the orientation and/or configuration (e.g., curvature) can be controlled by one or more actuators, which are typically located on the catheter's control handle, that in turn actuate a deflection mechanism (e.g., a pair of sliding members that move in opposition to each other within the catheter's control handle).

Although various configurations are known, deflection at the distal end of the medical device is often achieved through the use of one or more pull wires secured to the deflection mechanism at their proximal ends and to the distal end of the catheter (e.g., via a pull ring embedded in the wall of the medical device) at their distal ends. Manipulation of the actuator places certain pull wires in tension through movement of the deflection mechanism, which, in turn, effects change in the orientation and/or configuration of the distal end of the catheter.

In many extant steerable medical devices, the inner liner of the medical device shaft includes a multi-lumen configuration, with a larger central lumen and one or more smaller satellite lumens positioned about the periphery of the central lumen. The pull wires are typically routed through the satellite lumens.

### BRIEF SUMMARY

The instant disclosure provides a shaft for an elongate medical device. The shaft includes: a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens; and an outer layer bonded to the inner liner.

In certain embodiments of the disclosure, the wall thickness of the inner liner is uniformly between 0.004 inches and 0.006 inches.

The shaft can also include one or more pull wires, each of the one or more pull wires disposed within a respective satellite lumen of the one or more satellite lumens. For instance, a pair of satellite lumens can be positioned 180 degrees apart from each other about the periphery of the central lumen, or four satellite lumens can be positioned 90 degrees part from each other about the periphery of the central lumen.

An optional reinforcing layer may be disposed between the inner liner and the outer layer.

Also disclosed herein is an elongate medical device including a shaft having a distal end and a proximal end. The shaft includes: a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens; an outer layer bonded to the inner liner; and one or more pull wires disposed within the one or more satellite lumens, a distal end of each of the one or more pull wires secured to the distal end of the shaft. The medical device further includes a handle attached to the proximal end of the shaft, the handle including an actuator and a deflection mechanism, wherein a proximal end of each of the one or more pull wires is operably coupled to the actuator through the deflection mechanism such that actuation of the actuator places at least one pull wire of the one or more pull wires in tension to thereby deflect the distal end of the shaft.

The one or more satellite lumens may be a pair of satellite lumens positioned 180 degrees apart from each other about the periphery of the central lumen, and there may be a pair of pull wires respectively disposed within the pair of satellite lumens. In other embodiments, the one or more satellite lumens may be four satellite lumens positioned 90 degrees apart from each other about the periphery of the central lumen, and there may be four pull wires respectively disposed within the four satellite lumens.

It is contemplated that the wall thickness of the inner liner may uniformly be between 0.004 inches and 0.006 inches.

An optional reinforcing layer may be present between the inner liner and the outer layer.

The instant disclosure also provides a method of manufacturing an elongate medical device. The method includes: forming a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens; forming an outer layer around the inner liner; and bonding the outer layer to the inner liner, thereby forming the elongate medical device.

In some embodiments of the disclosure, the step of bonding the outer layer to the inner liner includes reflow bonding the outer layer to the inner liner.

One or more pull wires may be disposed within the one or more satellite lumens, wherein a distal end of each of the one or more pull wires is secured to a distal end of the elongate medical device. The method can also include: attaching a proximal end of the elongate medical device to a handle, the handle including an actuator and a deflection mechanism operably coupled to the actuator; and securing a proximal end of each of the one or more pull wires to the deflection mechanism.

In some embodiments, of the disclosure, the one or more satellite lumens includes a pair of satellite lumens positioned 180 degrees apart from each other about the periphery of the central lumen, and the one or more pull wires includes a pair of pull wires respectively disposed within the pair of satellite lumens. In other embodiments, the one or more satellite lumens includes four satellite lumens positioned 90 degrees apart from each other about the periphery of the central lumen, and the one or more pull wires comprises four pull wires respectively disposed within the four satellite lumens.

The wall thickness of the inner liner may uniformly be between 0.004 inches and 0.006 inches.

A reinforcing layer may optionally be formed between the inner liner and the outer layer prior to bonding the outer layer to the inner liner.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of an exemplary steerable catheter according to embodiments of the instant disclosure.

FIG. 2 depicts the handle of the catheter of FIG. 1 with the exterior housing removed in order to visualize certain internal components thereof.

FIG. 3 illustrates components of a shaft assembly according to embodiments of the instant disclosure.

FIG. 4 depicts the shaft assembly of FIG. 3 after bonding.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure relates to a shaft for an elongate medical device and a method of its manufacture. The shaft described herein may be employed to good advantage in the construction of various medical devices, and, in particular, in the construction of various catheters. For the sake of illustration, certain embodiments of the disclosure will be explained herein with reference to the construction of a steerable intracardiac echocardiography (ICE) catheter, such as the ViewFlex™ Xtra ICE catheter (Abbott Laboratories; Abbott Park, Illinois). Those of ordinary skill in the art will understand, however, how to apply the teachings herein to good advantage in other contexts and/or with respect to other devices (e.g., steerable diagnostic and therapeutic catheters, steerable introducers, and the like).

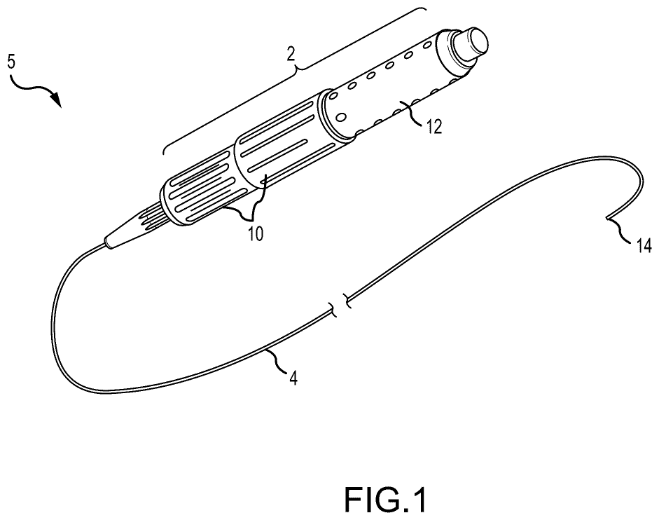

FIG. 1 depicts an exemplary steerable catheter 5. Catheter 5 generally includes a flexible tubular shaft (or body) 4 and a handle 2. It should be understood that the overall length of shaft 4 as depicted in FIG. 1 is merely exemplary and may vary consistent with the teachings herein, provided, of course, that it is sufficient to reach the intended destination within the patient's body (e.g., the heart).

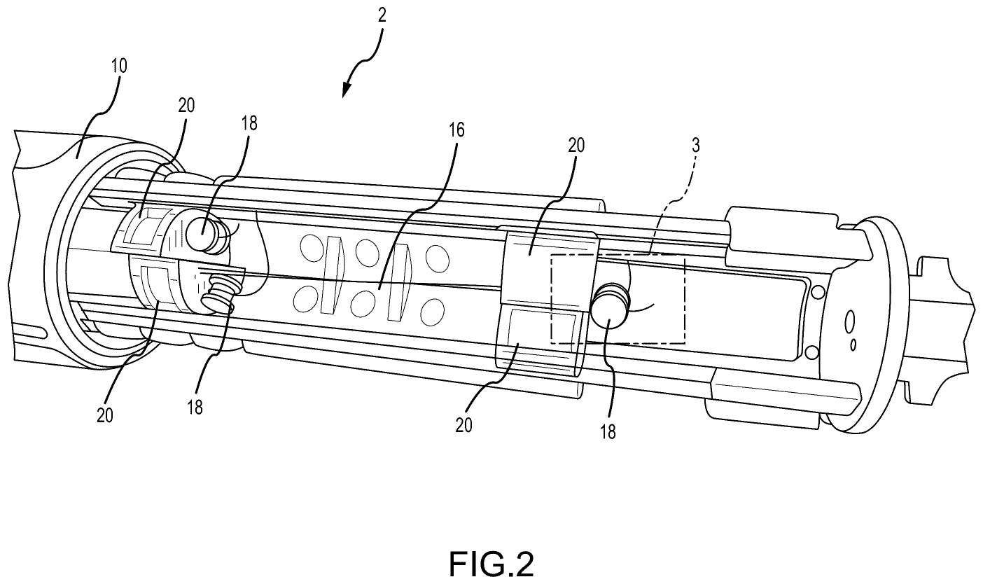

As shown in FIG. 1, handle 2 is connected to shaft 4 and includes actuators 10 and a grip portion 12. As those of ordinary skill in the art will appreciate, manipulation of actuators 10 (e.g., rotating actuator 10 in one direction or the other about the longitudinal axis of handle 2) causes distal end 14 of body 4 to deflect through the action of one or more pull wires (visible, for example in FIG. 2) extending from within handle 2 to a point within or near distal end 14 of shaft 4, where they may be secured to shaft 4 via a pull ring or other suitable structure.

In FIG. 2, grip portion 12 of handle 2 has been removed, thus making visible a plurality of pull wires 16 within handle 2. Each pull wire 16 is secured to a wire lock 18, which in turn can ride on a respective slider block 20. Thus, as those of ordinary skill in the art will recognized, rotating actuator 10 in one direction or the other will cause the members of a pair of slider blocks 20 to move in opposing directions (that is, one slider block 20 of the pair will move proximally, while the other slider block 20 of the pair will move distally), such as by action of a rack-and-pinion gearing system within handle 2. Slider block 20 moving proximally will push wire lock 18 riding thereon proximally, which places the corresponding pull wire 16 secured thereto into tension, in turn deflecting distal end 14 of shaft 4.

Insofar as the basic construction and operation of steerable catheters will be familiar to those of ordinary skill in the art, they need not be, and will not be, discussed in detail herein, except as necessary to understand the instant disclosure. By way of illustration only, however, U.S. Pat. Nos. 8,734,699, 8,676,290 and 7,691,095, as well as United States patent application publication no. 2007/0299424, all of which are hereby incorporated by reference as though fully set forth herein, describe various exemplary steerable catheters 5 and/or handles 2 in connection with which the instant teachings may be applied to good advantage.

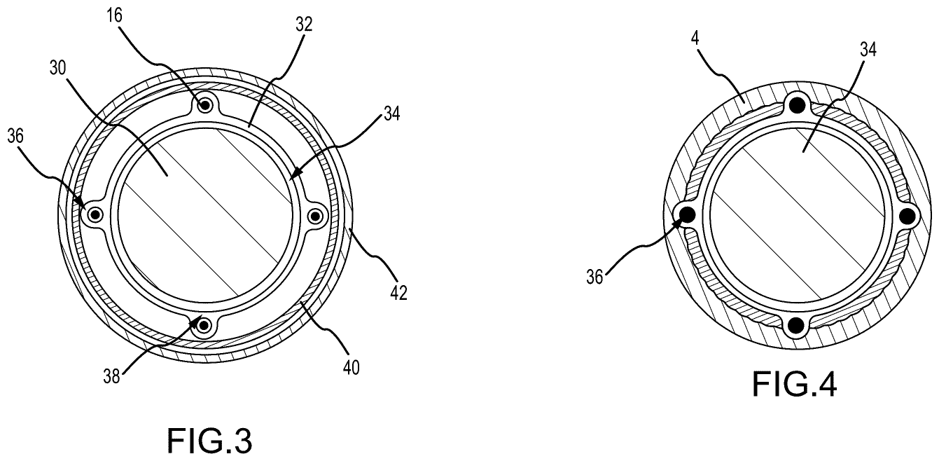

One method of manufacture of catheter 5, and in particular of at least a portion of shaft 4, according to an embodiment of the present disclosure will be described with reference to FIG. 3. As they are assembled, the components of shaft 4 will be referred to collectively as a “shaft assembly.”

As depicted in FIG. 3, a mandrel or hypotube 30, which can be round in cross-section and from about 6 inches to about 4 feet in length, may be the first component of the shaft assembly during manufacture of shaft 4. Typically, mandrel 30 is disposable. Mandrel 30 has a distal end and a proximal end.

An inner liner 32 is formed about mandrel 30. For example, inner liner 32 may be knotted at one end (e.g., the distal end) and then fed onto mandrel 30. In other aspects of the disclosure, inner liner 32 may be extruded about mandrel 30.

Inner liner 32 may be an extruded polymeric tubing, such as pre-extruded (and optionally chemically-etched) polytetrafluoroethylene (PTFE) tubing (e.g., TEFLON® brand tubing). Inner liner 32 may also be made of other melt-processable polymers, including, without limitation, fluorinated ethylene-propylene copolymer (FEP), perfluoroalkoxyethylene (PFA), poly (vinylidene fluoride), poly (ethylene-co-tetrafluoroethylene), and other fluoropolymers either with or without surface treatments such as chemical etching, plasma and corona treatment, and the like.

One of ordinary skill will also appreciate that inner liner 32 may be made of some melt-processable thermoplastic elastomeric polymers with sufficiently high mechanical strength and rigidity (e.g., durometer of at least about 60 D), including, without limitation, polyamide-based thermoplastic elastomers (namely poly (ether-block-amide), Pebax®), polyester-based thermoplastic elastomers (e.g., Hytrel®), thermoplastic polyurethanes (e.g., Pellethane®, Estane®), ionic thermoplastic elastomers, functionalized thermoplastic olefins and any combinations thereof.

In general, suitable materials for inner liner 32 may also be selected from various thermoplastics, including, without limitation, polyamides, polyurethanes, polyesters, functionalized polyolefins, polycarbonate, polysulfones, polyimides, polyketones, liquid crystal polymers and any combination thereof. Specific suitable materials for inner layer 26 include, without limitation, Pebax® 7233, Pebax® 6333, Grilamid L25, Rilsan AESNO, Rilsan BESNO, Makrolon 3108, Makrolon 1239, and the like.

According to embodiments disclosed herein, inner liner 32 is a unitary structure (that is, inner liner 32 is of one-piece construction, such as a single, continuous extrusion along its length) that defines several lumens. These lumens generally include a central lumen 34 and one or more satellite lumens 36 positioned about the periphery of central lumen 34. As shown in FIG. 3, central lumen 34 contains mandrel 30 during manufacture, while satellite lumens 36 contain pull wires 16.

For purposes of illustration, four satellite lumens 36 and corresponding pull wires 16, spaced about 90 degrees from each other around central lumen 34, are shown in FIG. 3. As those of ordinary skill in the art will recognize, this configuration of orthogonal, diametrically-opposed pairs of pull wires 16 permits multilateral deflection of the distal end 14 of catheter 5 in substantially any plane.

Of course, the configuration of FIG. 3 is merely exemplary, and other configurations are contemplated. For example, inner liner 32 can include single a pair of diametrically-opposed satellite lumens 36 and corresponding pull wires 16. This configuration permits bilateral deflection of the distal end 14 of catheter 5 in a single plane.

To improve retention of pull wires 16 within satellite lumens 36 and prevent them from cutting or tearing through the wall of inner liner 32 into central lumen 34 when their opposing members are under tension, it is desirable for the wall of inner liner 32 to be sufficiently thick where satellite lumens 36 are separated from central lumen 34 (e.g., regions 38). Yet, on the other hand, the wall of inner liner 32 within regions 38 should not be so thick as to undesirably impair the deflectability of catheter 5.

In embodiments of the disclosure, the wall of inner liner 32 is between about 0.004 inches and about 0.006 inches thick within regions 38. Indeed, the wall of inner liner 32 may uniformly be between about 0.004 inches and about 0.006 inches thick. It is also contemplated, however, that the wall thickness of inner liner 32 may vary.

One or more optional reinforcing layers 40 may then be formed about inner liner 32. Those of ordinary skill in the art will be familiar with the use of reinforcing layers 40, including reinforcing layers 40 made of braided or wound reinforcing wires, non-metallic fibers, or the like, in connection with the construction of catheter shafts.

Reinforcing layer(s) 40 may be formed separately on a disposable core and slipped about inner liner 32. Alternatively, reinforcing layer(s) may be formed directly upon inner liner 32 (e.g., braided about inner layer 32 using a braiding machine) to form a reinforced inner liner.

Insofar as various configurations for reinforcing layer(s) 40 will be familiar to those of ordinary skill in the art, a detailed description of specific configurations of reinforcing layer(s) 40 is not necessary to an understanding of the instant disclosure. Nonetheless, for purposes of illustration, several exemplary configurations for reinforcing layer(s) 40 are disclosed in U.S. Pat. Nos. 8,647,323 and 8,431,057, both of which are hereby incorporated by reference as though fully set forth herein.

One or more outer layers 42 are formed about reinforcing layer(s) 40 (or inner liner 32, if no reinforcing layer 40 is present). It should be understood that any number of substantially concentrically disposed outer layers 42 can be used without departing from the scope of the instant disclosure. Likewise, each outer layer 42 may be one continuous longitudinal segment or a plurality of shorter segments abutting each other along the length of the shaft assembly.

In some embodiments, outer layer(s) 42 are extruded about reinforcing layer(s) 40 (or inner liner 32, if no reinforcing layer 40 is present). In other embodiments of the disclosure, outer layer(s) 42 are separately extruded and then slipped about the shaft assembly.

Outer layer(s) 42 are typically melt-processable polymeric tubes, such as extruded polytetrafluoroethylene (PTFE) tubing (e.g., Teflon® brand tubing), optionally including surface etching. Outer layer(s) 42 may also be made of other melt processing polymers, including, without limitation, etched polytetrafluoroethylene and other fluoropolymers, poly (ether block amide) s, thermoplastic polyurethanes, polyester-based thermoplastic elastomers, and other thermoplastic elastomers. One such elastomer is Pebax®, made by Arkema, Inc. Pebax of various durometers may be used, including, without limitation, Pebax 30 D to Pebax 72 D.

Of course, one of ordinary skill in the art will recognize that various thermoplastics described as suitable for use as inner liner 32 (e.g., polyamides, polyesters, polycarbonate, polyurethane, polyolefins, polysulfones, polyimides, liquid crystal polymers, aromatic polyketones, and the like) are also generally suitable for use as outer layer(s) 42. One of ordinary skill will also appreciate that the material of outer layer(s) 42 may be different from or the same as the material of inner liner 32, and/or from each other, as desired, and will further appreciate how to select suitable materials for inner liner 32 and outer layer(s) 42 for a particular application of catheter 5.

The thickness of inner liner 32 and outer layer(s) 42 may be the same or different. Further, in some embodiments, it may be desirable for there to be at least partial chemical compatibility between inner liner 32 and outer layer(s) 42. This may promote bonding between the layers of the shaft assembly and reduce the likelihood of strain-induced polymer delamination under manipulation of catheter 5. Such compatibility may be provided by forming inner liner 32 and outer layer(s) 42 of materials whose polarity and/or solubility parameter are similar to each other. Alternatively, or additionally, chemical modifications may be undertaken to achieve at least partial chemical compatibility between these polymeric layers. In other embodiments, one or more of inner liner 32 and outer layer(s) 42 may include a coupling agent, such as silanes, zirconates, titanates, and the like. In still other embodiments, one or more of inner liner 32 and outer layer(s) 42 may include polymeric modifiers or adhesion promoters.

FIG. 3 depicts a cross section of the shaft assembly including inner liner 32, reinforcing layer 40, and a single outer layer 42 before forming catheter shaft 4, which may be accomplished by lamination of the various layers by heating (e.g., reflow bonding). In some embodiments, a layer of heat shrink is placed over outermost outer layer 42. Heat shrink may be a fluoropolymer or polyolefin material such as polytetrafluoroethylene (PTFE) or fluorinated ethylene-propylene copolymer (FEP).

As an alternative to a heat shrink, the shaft assembly may be placed into a suitable mold prior to subsequent processing. Either heat shrink or a suitable mold may be generally referred to as a “shape retention structure,” so named because it retains the overall shape of the shaft assembly (that is, the generally circular transverse cross-section) during melt-processing.

The shaft assembly may then be melt-processed. Energy (e.g., radiofrequency energy or thermal energy) is applied to the shaft assembly, for example to the outer surface of the shaft assembly, to bond inner liner 26 and outer layer(s) 42 together in a process often referred to as “reflow bonding.” The heat shrink has a higher melting or softening temperature than inner liner 32 and outer layer(s) 42 such that, during the melting process, the heat shrink will contract while retaining its tubular shape. The combination of applied energy and pressure exerted by the heat shrink forces melted inner liner 32 and outer layer(s) 42 to flow and redistribute about the circumference of the shaft assembly and bond together, encapsulating reinforcing layer(s) 40 therebetween (if present).

Once the shaft assembly has cooled, mandrel 30 can be removed, leaving central lumen 34 extending through at least a portion of formed catheter shaft 4. Optionally, the heat shrink tube may also be removed, such that outermost outer layer 42 becomes the outermost layer of catheter shaft 4 (though, as a result of the reflow bonding process, the wall of catheter shaft 4 can generally be considered unitary, with no readily-discernable interface between former inner liner 32 and former outer layer(s) 42, particularly where both inner layer 32 and outer layer(s) 42 were of the same material in the first instance. FIG. 4 depicts the shaft assembly after the conclusion of the reflow bonding process (that is, FIG. 4 depicts a transverse cross-section of catheter shaft 4 formed according to an embodiment of the instant disclosure).

Although several embodiments have been described above with a certain degree of particularity, those skilled in the art could make numerous alterations to the disclosed embodiments without departing from the spirit or scope of this invention.

For example, pull wires 16 may be inserted into satellite lumens 36 either before or after reflow bonding.

In addition, it is contemplated that a catheter or other elongate medical device according to the teachings herein may be manufactured using alternative techniques. For example, rather than bonding the layers of the shaft assembly via melt-processing (e.g., reflow bonding) as generally described above, one or more layers may be extruded over one another (e.g., extrusion of outer layer 42 over reinforcing layer 40). Where one or more layers are extruded, they may be coextruded.

It is also contemplated to utilize a combination of reflow bonding and extrusion processes.

As another example, the various polymeric layers may be formed by wrapping or winding a suitable material about the catheter shaft assembly (e.g., wrapping surface-etched PTFE tape about mandrel 30 to form inner layer 32).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A shaft for an elongate medical device, the shaft comprising:
a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens; and
an outer layer bonded to the inner liner.

2. The shaft according to claim 1, wherein the wall thickness of the inner liner is uniformly between 0.004 inches and 0.006 inches.

3. The shaft according to claim 1, further comprising one or more pull wires, each of the one or more pull wires disposed within a respective satellite lumen of the one or more satellite lumens.

4. The shaft according to claim 1, wherein the one or more satellite lumens comprises a pair of satellite lumens positioned 180 degrees apart from each other about the periphery of the central lumen.

5. The shaft according to claim 1, wherein the one or more satellite lumens comprises four satellite lumens positioned 90 degrees part from each other about the periphery of the central lumen.

6. The shaft according to claim 1, further comprising a reinforcing layer disposed between the inner liner and the outer layer.

7. An elongate medical device comprising:
a shaft having a distal end and a proximal end, the shaft comprising:
a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens;
an outer layer bonded to the inner liner; and
one or more pull wires disposed within the one or more satellite lumens, a distal end of each of the one or more pull wires secured to the distal end of the shaft; and
a handle attached to the proximal end of the shaft, the handle including an actuator and a deflection mechanism, wherein a proximal end of each of the one or more pull wires is operably coupled to the actuator through the deflection mechanism such that actuation of the actuator places at least one pull wire of the one or more pull wires in tension to thereby deflect the distal end of the shaft.

8. The elongate medical device according to claim 7, wherein the one or more satellite lumens comprises a pair of satellite lumens positioned 180 degrees apart from each other about the periphery of the central lumen, and wherein the one or more pull wires comprises a pair of pull wires respectively disposed within the pair of satellite lumens.

9. The elongate medical device according to claim 7, wherein the one or more satellite lumens comprises four satellite lumens positioned 90 degrees apart from each other about the periphery of the central lumen, and wherein the one or more pull wires comprises four pull wires respectively disposed within the four satellite lumens.

10. The elongate medical device according to claim 7, wherein the wall thickness of the inner liner is uniformly between 0.004 inches and 0.006 inches.

11. The elongate medical device according to claim 7, wherein the shaft further comprises a reinforcing layer between the inner liner and the outer layer.

12. A method of manufacturing an elongate medical device, the method comprising:
forming a unitary inner liner defining a central lumen and one or more satellite lumens positioned about a periphery of the central lumen, wherein a wall thickness of the inner liner is between 0.004 inches and 0.006 inches where the inner liner separates the central lumen from each of the one or more satellite lumens;
forming an outer layer around the inner liner; and
bonding the outer layer to the inner liner, thereby forming the elongate medical device.

13. The method according to claim 12, wherein bonding the outer layer to the inner liner comprises reflow bonding the outer layer to the inner liner.

14. The method according to claim 12, further comprising disposing one or more pull wires within the one or more satellite lumens, wherein a distal end of each of the one or more pull wires is secured to a distal end of the elongate medical device.

15. The method according to claim 14, further comprising:
attaching a proximal end of the elongate medical device to a handle, the handle including an actuator and a deflection mechanism operably coupled to the actuator; and
securing a proximal end of each of the one or more pull wires to the deflection mechanism.

16. The method according to claim 14, wherein the one or more satellite lumens comprises a pair of satellite lumens positioned 180 degrees apart from each other about the periphery of the central lumen, and wherein the one or more pull wires comprises a pair of pull wires respectively disposed within the pair of satellite lumens.

17. The method according to claim 14, wherein the one or more satellite lumens comprises four satellite lumens positioned 90 degrees apart from each other about the periphery of the central lumen, and wherein the one or more pull wires comprises four pull wires respectively disposed within the four satellite lumens.

18. The method according to claim 12, wherein the wall thickness of the inner liner is uniformly between 0.004 inches and 0.006 inches.

19. The method according to claim 12, further comprising forming a reinforcing layer between the inner liner and the outer layer prior to bonding the outer layer to the inner liner.
